(19) United States
(12) Patent Application Publication
(10) Pub. No.: US 2007/0111229 A1
(43) Pub. Date: May 17, 2007

(54) Pathogenicity determinants which can be used as targets for developing means for preventing and controlling bacterial infections and/or systemic dissemination

(76) Inventor: Sonia Escaich, Paris (FR)

Correspondence Address: NIXON & VANDERHYE, PC, 901 NORTH GLEBE ROAD, 11TH FLOOR, ARLINGTON, VA, 22203, US

(21) Appl. No.: 11/506,821

(22) Filed: Aug. 21, 2006

Related U.S. Application Data
(62) Division of application No. 10/520,820, filed on Apr. 28, 2005, now abandoned.

(30) Foreign Application Priority Data
Jul. 9, 2002 (FR) 02 08 636

(51) Int. Cl. C12Q 1/68; C07H 21/04; C12N 15/74

(52) U.S. Cl. 435/6; 435/471; 435/252.3; 536/23.7

(57) ABSTRACT

The invention relates to a method for identifying and selecting a gene required for the proliferation in vivo of a pathogenic microorganism, comprising: using a strain of the pathogenic microorganism, generating mutants for inactivation in the genes encoding these factors, determining the virulence of these mutants on an expiremental model of infection, and their effect on enteric colonization in an axenic mouse model, and selecting the bacterial genes essential for resistance to serum in vitro, and essential, in the host, for dissemination in the serum. Application to the screening of compounds which inhibit the products of the genes identified, and to the inhibition in vitro of the proliferation of a pathogenic microorganism in serum.

The present application is a divisional of Ser. No. 10/520,820 filed Apr. 28, 2005 (pending) which is 371 U.S. national phase of PCT/EP03/08209 filed Jul. 9, 2003, which designated the U.S. and claims benefit of FR 0208636 filed Jul. 9, 2002, the entire contents of each of which is hereby incorporated herein by reference.

The invention relates to pathogenicity determinants which can be used as targets for developing means for preventing and controlling bacterial infections and/or systemic dissemination.

Current treatments for infectious diseases of bacterial origin are based on the inhibition of essential bacterial targets in vitro using antibiotics. These targets are conserved in many bacterial species and make it possible to treat various types of infection. However, broad-spectrum antibiotics are active on the host's commensal flora, which promotes the acquisition and transfer of mechanisms of resistance to these antibiotics, hence a limiting of the effectiveness of current treatments with antibiotics. A need therefore exists for novel antibacterial treatments.

In this regard, the invention provides a novel strategy, the aim of which is to specifically target pathogenic bacteria without significantly altering their growth at their portal of entry into the host organism, where they are in a situation of commensalism. These pathogens are in particular the bacteria responsible for serious systemic infections, such as E. coli, in general Enterobacteria, Pseudomonas, Acinetobacter, Moraxella and Neisseria and, for the gram positives, the bacteria of the genus Staphylococcus, Enterococcus and Streptococcus.

It is known, specifically, that the bacteria responsible for serious infections are capable of growth in the presence of serum and are resistant to the bactericidal action of complement. This resistance allows dissemination of the infection, via the blood, to the various tissues of the host's body.

The ability of bacteria to grow in human serum is due to different pathogenicity/virulence factors. Among those frequently cited, mention will be made of the physical barrier, represented by the capsule, for access of complement to the bacterial membrane, the sialic acids of the capsule or of the O antigen which promote binding of factor H to c3b, and particular surface proteins such as PorA (Neisseria gonorrhoeae), YadA (Yersinia pestis) or protein M (Streptococcus pyogenes), which bind factor H, all these factors preventing complement activation.

Other proteins expressed or bound by the pathogens have proved to be important for resistance to complement and cause cleavage of complement factors or inhibit their binding to the surface of the bacterium (Rautemaa R.; Meri S., Microbes and Infection 1999, 1:785:794).

The lipopolysaccharide (LPS) of gram-negative bacteria is known to be a virulence factor, but the role of its various constituents on the resistance to serum has not been established for all bacterial species. For example, in some studies in E. coli, the O antigen is considered to be determinant (Burns S. M. Hull S. I. Infect Immun, 1998, September 66(9):4244-53); in other studies, the O antigen is thought to be less determinant than the capsular antigens for resistance to serum (Russo T. et al., Infect Immun, 1995, April 63(4):1263-9). Furthermore, the importance of these factors on intestinal colonization is unknown.

The inventors have carried out a systematic analysis of mutants for inactivation of the genes required for surface polysaccharide synthesis, and have demonstrated, in Escherichia coli strains responsible for extra-intestinal infections, EXPEC, which genes are essential for the resistance to serum and the dissemination in the blood. These results are based on the study of the effect of mutations on virulence and intestinal colonization in an animal model.

The invention is therefore directed towards a novel methodology for defining the targets required for virulence, and not essential in vitro, and thus providing novel anti-infectious agents specific for pathogenic bacteria, in particular for extra-intestinal E. coli, responsible for severe infections, as well as Gram positive strains, such as Streptococcus agalactiae. It is also directed towards the products of the genes required for resistance in the serum and virulence in vivo.

The method of the invention for identifying and selecting a gene required for the proliferation in vivo of a pathogenic microorganism is characterized in that it comprises:
using a strain of the pathogenic microorganism,
generating mutants for inactivation in the genes encoding the virulence factors,
determining the virulence of these mutants on an experimental model of infection and their effect on enteric colonization in an axenic mouse model, and
selecting the bacterial genes essential for resistance to serum in vitro and essential, in the host, for dissemination in the blood.

The pathogenic microorganism is in particular an EXPEC strain of E. coli or a Streptococcus agalactiae strain.

The virulence gene inactivation mutants used in this method fall within the scope of the invention.

Said mutants are characterized by the following properties: they are sensitive to serum; they are avirulent in mice model and they are able to colonize gut of axenic mice.

The invention is also directed towards the pathogenicity or virulence factors encoded by nucleic acids thus identified, which are necessary for the dissemination via the blood, but do not significantly affect the intestinal or mucosal colonization of pathogenic bacteria such as E. coli, Salmonella typhimurium, Klebsiella pneumoniae, Yersinia pestis, Serratia marcescens, Haemophilus influenzae, Pasteurella multocida, Vibrio cholerae, Pseudomonas aeruginosa, Acetinobacter, Moraxella catarrhalis, Burkholderia pseudomallei, Neisseria meningitidis, Neisseria gonorrhoeae, Campylobacter jejuni, Helicobacter pylori, Bacteroides fragilis, Clostridium acetobutylicum, Mycobacterium tuberculosis, Streptococcus pyogenes, Streptococcus agalactiae, Staphyloccus aureus and Enterococcus.

The invention is in particular directed towards the pathogenicity or virulence targets encoded by isolated or purified nucleic acids having sequences SEQ ID Nos 16-30.

The pathogenicity or virulence targets of the invention are more particularly encoded by nucleic acids having sequences SEQ ID Nos 16, 17, 19-30.

Said nucleic acids are cDNAs or RNAs.

It particularly relates to pathogenicity or virulence targets encoded by nucleic acids of E. coli.

In another embodiment of the invention, the pathogenicity or virulence targets are encoded by nucleic acids of Streptococcus agalactiae.

The invention is also directed towards the vectors comprising at least a nucleic acid coding for a pathologenicity or virulence target such as above defined and also the host cells containing at least one vector under the control of a suitable promoter.

The invention is also directed towards pathogenicity or virulence factors corresponding to isolated or purified polypeptides or peptides having one of the amino acid sequences SEQ ID Nos 1-15.

It more particularly relates to pathogenicity or virulence factors corresponding to isolated or purified polypeptides or peptides having the amino acid sequences SEQ ID Nos 1, 2, 4-15.

The antibodies which are capable of binding specifically to the peptides and polypeptides corresponding to said factors are also part of the invention.

These nucleic acids and peptides or polypeptides constitute targets for identifying compounds with a specific inhibitory effect on the systemic dissemination of a bacterial infection, and not on mucosal colonization or, for enterobacteria, on intestinal colonization, which makes it possible to preserve the commensal flora and to avoid the selection of resistance to the compounds.

The invention is thus directed towards the method for inhibiting the proliferation of a pathogenic microorganism in serum, comprising the use of an effective amount of a compound capable of inhibiting the activity, or of reducing the amount, of a nucleic acid as defined above, or of a compound capable of inhibiting the activity of a polypeptide or of a peptide as defined above.

It is also directed towards a method for screening compounds capable of inhibiting the expression of these nucleic acids or of the corresponding polypeptides and peptides, comprising bringing them into contact with the test compound, demonstrating the possible effect of the compound on their activity, and selecting the active compounds.

It is also directed towards a method for screening compounds capable of inhibiting the biochemical and/or enzyme activity of the polypeptides and peptides expressed by said nucleic acids.

The compounds thus selected are used, in accordance with the invention, to produce medicinal products for inhibiting a bacterial infection, in particular an extra-intestinal infection in the case of enterobacteria.

The invention thus provides a novel strategy and novel means for preventing or treating systemic bacterial dissemination, bacteraemia and septicaemia.

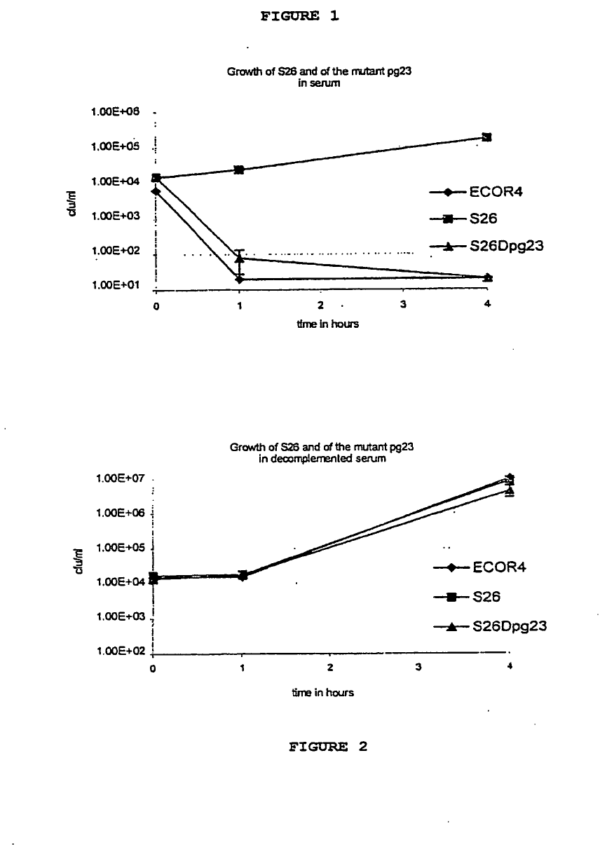

Other characteristics and advantages of the invention will be given in the following examples, with reference to FIGS. 1 to 3 and tables 1 to 5, said figures representing, respectively,

FIG. 1, the growth of S26 and of the mutant pg23 in serum,

FIG. 2, the growth of S26 and of the mutant pg23 in decomplemented serum,and

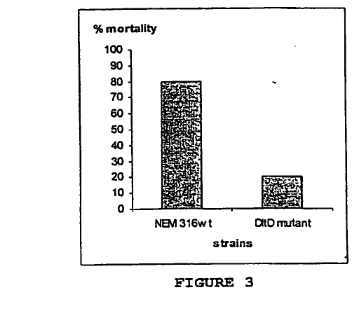

FIG. 3, the virulence of the DltD mutant of S. agalactiae.

### EXAMPLE 1

### Gene Corresponding to SEQ ID N^{o} 23

1—Inactivation of the Gene of Interest

The general strategy, based on a recombination system, consists in interrupting a gene, by allelic recombination, with a gene for selection (a gene for resistance to antibiotic in the present case) carried by a linear DNA fragment.

Initially, a plasmid is introduced into the bacterium (for example E. coli), so as to introduce, in trans, the proteins which will induce the recombination. The plasmid carrying an ampicillin resistance gene is thermosensitive (30° C.), which will make it possible to easily eliminate it after use in the bacterium.

The plasmid is introduced into the bacterium by electroporation. After electroporation, the ampicillin-resistant bacteria will be those which have integrated the plasmid, and will be selected. This step is entirely carried out at 30° C., the permissive temperature for the plasmid.

Synthesis of the PCR Fragment Specific for the Target Gene (pg23)

A PCR is carried out, on a matrix plasmid carrying the selection gene (chloramphenicol resistance), using primers pg23P1 and pg23P2 of sequences SEQ ID No 31 and SEQ ID No 32, respectively, made up of two parts:
in 3′: 20 bp homologous to the selection gene (chloramphenicol resistance): P1 or P2

in 5′: 40 bp homologous to the target gene (pg23): H1 or H2

```
pg23P1:
5′ TCGTGCAGGCCAACCTGCACAACAGAGTGATTTGATTAACGTGTAGG
                                                      H1
CTGGAGCTGCTTC 3′
    P1
Pg23P2:
5′ CAGGGTGCTGGCGCTCACCATTTCCGGAGACAGCTTAGACACATATG
                                                      H2
AATATCCTCCTTA 3′
    P2
```

A DNA fragment consisting of the selection gene (CAT: Chloramphenicol Acetyl Transferase) flanked by the regions homologous to the target gene H1 and H2 is thus obtained.

Step for Inactivation of the Target Gene

The bacterium containing the plasmid is cultured in LB medium at 30° C. with shaking, in the presence of 100 mM ampicillin and of 1 mM L-arabinose so as to induce the recombination system. When the bacteria are in the exponential growth phase (OD₆₀₀ₙₘ=0.5), the culture is stopped, and the bacteria are harvested and made electrocompetent. The PCR product specific for the target gene (pg23) is introduced into the bacterium by electroporation. The bacteria are then cultured in a non-selective rich medium (SOC medium) at 37° C. with shaking for 2 hours, and then plated out onto selective LB agar medium. After 18 hours at 37° C., only the bacteria which have integrated the gene for resistance to the antibiotic will have grown.

Verification of the Insertion of the Resistance Cassette

In order to verify the insertion of the resistance cassette, PCR reactions are carried out directly using colonies. Three pairs of primers are used: a pair in which the primers FR1 and FR2 frame the target gene, and two pairs using a primer located inside the resistance cassette, the other primer being located either upstream or downstream of the target gene.

Isolation of the Mutated Bacteria and Elimination of the Plasmid

The colonies thus verified by PCR are successively re-isolated on selected medium, twice on non-selective medium and a final time on selective medium at 37° C. Finally, the selected bacteria are tested for sensitivity to ampicillin, which reflects the absence of the plasmid. Three clones are thus chosen for each type of mutant.

2 —Test for the Mutant with Respect to Resistance to the Bactericidal Activity of Serum

The serum used is of human origin. In each experiment, growth was also effected for the wild-type strain (S26, clinical isolate of E. coli particularly resistant to serum and virulent in mice) and a strain, ECOR4, lacking a capsule and lipopolysaccharide (LPS). The growths were effected in triplicate and in two different sera. The growths were effected in parallel in complemented and decomplemented (30 min at 56° C.) serum in order to verify that the effect observed was due only to the lytic action of complement.

Using a preculture of two hours in RPMI reference minimum medium, the bacteria are brought Canto contact with 100% serum, at a starting inoculum of 10⁴cfu/ml. Counts are then performed at times 0, 1 and 4 hours, by plating various dilutions out on LB agar medium in the presence or absence of antibiotic. After 18 hours at 37° C., the bacteria are counted and a growth curve is produced from the results obtained. These results are given in FIGS. 1 and 2.

In this example, the mutant Δpg23 exhibits considerable sensitivity to the serum: a difference from the wild-type strain of more than 2 log at 1 hour and of more than 4 log at 4 hours is in fact observed. In addition, the results obtained in decomplemented serum and with the strain ECOR4 in serum indicate that the effect observed is indeed due to the bactericidal action of complement.

3 —Study of the Virulence in a Mouse Animal Model

Preparation of the Inoculum

The wild-type mutated bacteria are isolated from the strain, stored at −80° C., on an LB agar dish with or without antibiotic, and incubated at 37° C. for 18 hours. A preculture is prepared in liquid medium. Using a 1/10th dilution in 10 ml of LB, the culture is regrown at 37° C. with shaking for 2 hours. After culturing for 2 hours, the OD₆₀₀ₙₘ is measured and various dilutions are prepared in physiological saline, so as to obtain the desired inoculum. For the wild-type strain S26, the LD₅₀ corresponds to an inoculum of 5×10⁵ cfu/mouse and the LD₁₀₀ corresponds to an inoculum of 1×10⁶ cfu/mouse.

Virulence Test

The mice (6-week-old Balb/c) are given an intraperitoneal injection and the bacterial solution injected represents a volume of 100 μl. Five mice are used per dose. For S26Δpg23, 4 inoculums were tested and the survival rate was measured after 24 and 48 hours post-injection. In each experiment, the study was carried out in parallel with the wild-type strain, the LD₅₀ of which is 5×10⁵ cfu/mouse.

The mutant S26Δpg23, injected at a dose equal to 10 times the LD₁₀₀, causes no mortality, the mutation of the pg23 gene in the E. coli strain K1 S26 is therefore responsible for a considerable decrease in the virulence.

4 —Study of the Intestinal Colonization in an Axenic Mouse Animal Model

The entire experiment is carried out in a sterile environment, with sterile instruments, in an isolator, and the mice are given sterile food.

Mice

These are 6- to 8-week-old axenic female mice of the C3H/He J line.

Four animals are used per bacterial strain.

Preparation of the Inoculum

The wild-type and mutated bacteria are isolated from the strain, stored at −80° C., on an LB agar dish with or without antibiotic, and incubated at 37° C. for 18 hours. After culturing the strain in liquid medium, various dilutions are prepared in physiological saline, so as to obtain an inoculum of 10⁷ cfu/ml.

Colonization Test

The bacterial inoculation is carried out orally. During the 24 hours preceding inoculation, the mice are deprived or water. They are then given a bacterial solution at 10⁷ cfu/ml to drink for 4 hours. The volume of drink is measured at 0 and 4 hours, and, on average, a mouse absorbs 5 ml of this bacterial solution. The faeces are then sampled at various times, and a bacterial count is performed, taking the faeces up in physiological saline and plating out various dilutions on an LB agar dish with and without antibiotic.

The results are given in table 1 herein below.

TABLE 1

CFU/mg faeces

| Time in hours | S26wt | S26Δpg23 |
|---|---|---|
| 0 | 0 | 0 |
| 4 | 6.85E+05 | 1.65E+05 |
| 25 | 1.86E+06 | 2.84E+06 |
| 118 | 8.34E+06 | 7.94E+06 |
| 456 | 4.14E+06 | 6.64E+06 |

For the wild-type strain S26, as well as for the mutant S26Δpg23, colonization in the intestine was stably established. No difference is observed between the wild-type strain and the mutant Δpg23. The colonization is confirmed on the final day by removing the intestine and counting the bacteria after grinding of this organ.

5 —Cloning and Expression of the Selected Polypeptide

The nucleic acid encoding the polypeptide is cloned into a prokaryotic expression vector such as pET-14b with an N-terminal poly-his tag, according to conventional cloning methods.

The recombinant plasmid is then used to transform the E. coli strain BL21. The transformed cells are selected in the presence of ampicillin and the colonies are isolated. They are then cultured in the presence of IPTG in order to induce expression of the protein. The clones producing the protein are cultured and the total proteins are extracted by cell lysis. The recombinant protein is purified with a histidine tag affinity column, according to the manufacturer's protocol.

The protein thus obtained is purified and used in vitro to measure its enzyme activity.

### EXAMPLE 2

### Serum Sensitivity and LD₅₀ Determination of Mutant Strains in the Mice Model of Infection

Said mutants were also compared to the wild type S26 E. coli strain for LD₅₀ determination in the mice model of infection. As presented in Table 2 below, the number of colony forming unit (cfu) counted after culture for four hours in serum was higher in the wild type (wt) S26 strain than in mutants indicating that mutants were sensitive to serum killing.

All the different mutants were either much less virulent in mice than the wild type strain as shown by the increase in LD₅₀ (lethal dose 50), or completely avirulent as no dose killing 50% of mice could be reach with the mutants.

TABLE 2
Serum sensitivity and virulence attenuation for E. coli K1 S26
mutants in the proteins corresponding to sequence number 1 to 13

| | Serum sensitivity | Virulence |
|---|---|---|
| Sequence | #Δlog (cfu/ml | attenuation |
| Number | serum) | *Δlog (LD50) |
| 1 | +4 | avirulent^{a} |
| 2 | +4 | +1 |
| 3 | +5 | +1 |
| 4 | +4 | +1 |
| 5 | +4 | +2.5 |
| 6 | +4 | +0.5 |
| 7 | +4 | +0.5 |
| 8 | +4 | avirulent^{a} |
| 9 | +1 | avirulent^{a} |
| 10 | +2 | avirulent^{a} |
| 11 | +4 | +2 |
| 12 | +4 | +2 |
| 13 | +4 | avirulent^{a} |

avirulent^{a}: no dose killing 50% of mice could be reach with that mutant.
#Δlog (cfu/ml serum) = log (cfu S26wt/ml serum) − log (cfu S26 mutant/ml serum) values obtained after 4 hours in serum
*Δlog (LD₅₀) = log (LD₅₀ S26mutant) − log (LD₅₀ S26wt) values obtained 48 hours after inoculation

The mutants of genes encoding the target proteins corresponding to sequences 1 to 13, which were attenuated for virulence, were still able to colonize the intestine of axenic mice as shown by persistence of bacteria in the faeces of the animals over a period of eight days. These results are prsented in Table 3.

TABLE 3
Gut colonization for E. coli K1 S26 wt and mutants in the proteins
corresponding to sequence number 1 to 13 in an axenic mouse model

| | |
|---|---|
| | Gut colonization |
| Sequence | cfu/mg faeces |

| | number | Day 1 | Day 8 |
|---|---|---|---|
| S26 wt | | *1.34 · 10⁶ | *5.29 · 10⁶ |
| S26 | 1 | 9.73 · 10⁵ | 2.51 · 10⁶ |
| mutants | 2 | 1.02 · 10⁶ | 6.85 · 10⁶ |
| | 3 | 1.44 · 10⁶ | 3.48 · 10⁶ |
| | 4 | 1.24 · 10⁶ | 1.65 · 10⁶ |
| | 5 | 1.15 · 10⁵ | 4.64 · 10⁵ |
| | 6 | 9.96 · 10⁵ | 3.51 · 10⁶ |
| | 7 | 2.40 · 10⁴ | 2.51 · 10⁶ |
| | 8 | 2.84 · 10⁶ | 6.64 · 10⁶ |
| | 9 | 1.80 · 10⁶ | 1.51 · 10⁶ |
| | 10 | 9.62 · 10⁵ | 2.24 · 10⁶ |
| | 11 | 2.72 · 10⁵ | 8.56 · 10⁵ |
| | 12 | 3.13 · 10⁵ | 9.09 · 10⁵ |
| | 13 | 5.91 · 10⁵ | 1.67 · 10⁶ |

*mean values based upon six experiments

The bacteria colonizing the intestine of axenic mice after eight days were characterized to verify that they correspond to the mutant strains that were inoculated orally.

The bacteria recovered from the faeces of animals had a phentype of chloramphenicol resistance and serum sensitivity, the chloramphenicol acetyl transferase gene inserted during the mutagenis could also be detected by PCR.

Mutations in genes encoding target proteins (sequence number 1 to 13) were still present in bacteria colonizing the intestine of axenic mice as shown in Table 4.

TABLE 4
Characterization of bacteria recovered from axenic mice after
intestinal colonization by mutants in genes encoding proteins
sequence 1 to 13

| Sequence | Serum sensitivity | *Mutant |
|---|---|---|
| Number | #ΔLog (cfu/ml serum) | genotype |
| 1 | +5 | Cm^{R}, PCR + |
| 2 | +4 | Cm^{R} |
| 3 | +5 | Cm^{R} |
| 4 | +3 | Cm^{R} |
| 5 | +5 | Cm^{R}, PCR + |
| 6 | +2 | Cm^{R} |
| 7 | +2 | Cm^{R} |
| 8 | Nd | Cm^{R} |
| 9 | +2 | Cm^{R} |
| 10 | +3 | Cm^{R} |
| 11 | +5 | Cm^{R}, PCR + |
| 12 | +4 | Cm^{R}, PCR + |
| 13 | +4 | Cm^{R}, PCR + |

#ΔLog (cfu/ml serum) = log (cfu S26wt/ml serum) − log (cfu S26mutant/ml serum) values obtained after 4 hours in serum
*The presence of the gene encoding the chloramphenicol acetyltransferase, inactivating the genes encoding the proteins of sequence number 1 to 13, has been verified by PCR and chloramphenicol resistance (Cm^{R}).

In conclusion, the results presented in this example demonstrate that genes encoding the enzymes involved in the LPS inner core metabolism are not essential in E. coli strains for colonization, but are necessary for resistance to complement and virulence in vivo.

They represent as such good targets for inhibitors that will selectively block bacterial replication in blood tissue.

### EXAMPLE 2

### Mutants of Protein SEQ ID N^{o} 14

The present invention relates to novel mutant strain of Group B Streptococcus (GBS) (Streptococcus agalactiae). In this particular example, the identified targets correspond to gene sequence number 29 encoding a protein sequence number 14 involved in incorporation of D-alanine residues into the cell wall-associated lipoteichoic acids (LTAs) in Gram+bacteria.

The gene sequence number 29 is homologous to the dltD gene found in other gram positive bacteria and is the last gene of the dlt operon. The Gram+bacterial model used is the pathogenic strain S. agalactiae NEM316. S. agalactiae is a bacterium commonly found in the human flora and is phylogeneticaly close to Gram+bacteria responsible for nosocomial septicemia.

The virulence of GBS mutants in the dlt operon is strongly impaired in mouse and newborn rat models.

Interestingly, the loss of virulence is presumably due to an increased sensitivity to antimicrobial cationic peptides, such as defensins, which are produced by numerous cells types in particular phagocytes.

The use of mutant of S. agalactiae, in which the dltD gene have been inactivated, demonstrates that the product of that gene is a good target for the development of inhibitors of virulence of S. agalactiae as well as against other Gram+pathogens.

Construction of a DtlD Mutant in Wild Type S. Agalactiae NEM316:

A mutant in the dltD gene was constructed from S. agalactiae NEM316 strain by inserting, using double cross-over, a kanamycin resistance cassette.

To construct DltD mutant of S. agalactiae NEM316, a promoterless and terminatorless kanamycin resistance cassette aphA-3 within DNA segment internal to dltD were inserted in the same direction of transcription. This was done by ligation after digestion with appropriate enzymes, of PCR products obtained by using the primers of SEQ ID N^{o} 33 and. 34 respectively,

SEQ ID No 33:
5′-CAGTGAATTCGCGTTGACGAAGGCAGG-3′,
and
SEQ ID No 34:
5′-GACGGGTACCATACCTATCGTAGGTTG-3′,
and the primers of SEQ ID No 35 and SEQ ID No
36, respectively,
SEQ ID No 35:
5′-AGTGGATCCACTACACAGGGCTTGATC-3′,
and
SEQ ID No 36:
5′-GACCTGCAGCCCTTGATTATCCCTATCC-3′.

A 0.4 kb dltD EcoRI-KpnI fragment was inserted into the thermosensitive shuttle vector pG+host5ΩaphA-3 (Biswas et al., 1993, J Bacteriol. 175:3628-3635) containing the kanamycin resistance cassette to generate pG1Ω EKaphA-3. A 0.8 kb closely spaced dltD region BamHI-PstI fragment was inserted into pG1Ω EKaphA-3 to generate pG1Ω EKaphA-3BP. The resulting vector was introduced by electroporation into NEM316. Transformants were selected on Todd-Hewitt (TH) agar plates containing 10 mg l⁻¹ erythromycin at 30° C. Allelic exchange was obtained at the non-permissive temperature (42° C.) by homologous recombination using a two-step procedure described previously (Biswas et al., 1993).

A double-crossover event between the homologous sequences resulted in nucleotides deletion and insertion of the kanamycine cassette. Recombinant bacteria containing this insertion deletion were selected for kanamycine resistance.

This chromosome disruption in the dltD gene was confirmed in one of the recombinant clones by sequencing the nucleotides of the mutant.

Sensitivity of the wild type S. agalactiae strain NEM316 and the DltD mutant to various antimicrobial peptides :

The sensitivity of wild type S. agalactiae NEM316 and DltD mutant to cationic antimicrobial peptides was measured by using a disk diffusion methods. The 2 strains were grown on blood agar plates and incubated for 18 hours at 37° C. Each strain was tested using colistin (50 μg) and polymixin (10 μg) disks. Sensitivity.or resistance of NEM316 strain and the DltD mutant to each compound was determined by the size of the growth inhibition area around disk.

The DltD mutant exhibited an increased sensitivity to the cationic antimicrobial peptides colistin, and polymyxin B as shown in table 5.

TABLE 5
Results of sensitivity to colistin and polymixin B of control
strains S. agalactiae NEM316 and DltD mutant

| | |
|---|---|
| Disc | Inhibition area (mm) |

| | content | | Mutant |
|---|---|---|---|
| | (μg) | NEM316 | DltD |
| Colistin | 50 | 0 | 14 |
| Polymixin B | 10 | 0 | 14 |

Study of Virulence in a Mouse Animal Model

We studied the role of DltD in the virulence of S. agalactiae. Groups of ten mice (six week-old Balb/c) were inoculated intravenously with 5×10⁷ bacteria. At 2 days post infection, 80% of mice infected with the wild type strain NEM316 died and only two deaths were recorded for mice infected with the DltD mutant. FIG. 1 illustrates the results obtained with the DltD defective GBS mutant. The result demonstrates that the product of the dltD gene is necessary for virulence of GBS in mice.

I claim:

1. Method for identifying and selecting a gene required for the proliferation in vivo of a pathogenic microorganism, comprising:
using a strain of the pathogenic microorganism,
generating mutants for inactivation in the genes encoding these factors,
determining the virulence of these mutants on an experimental model of infection, and their effect on enteric colonization in an axenic mouse model, and
selecting the bacterial genes essential for resistance to serum in vitro, and essential, in the host, for dissemination in the serum.

2. Method according to claim 1, characterized by the use of an E. coli strain EXPEC or a Streptococcus agalactie strain.

3. Mutant nucleic acids for inactivation of the virulence genes as implemented in the method according to claim 1.

4. Pathogenicity or virulence targets encoded by isolated or purified nucleic acids corresponding to one of the nucleotide sequences SEQ ID Nos 16-30.

5. Pathogenicity or virulence targets according to claim 4, wherein said nucleic acids correspond to one of the nucleotide sequences SEQ ID Nos 16,17, 19-30.

6. Pathogenicity or virulence targets according to claim 4, wherein said nucleic acids are cDNAs.

7. Pathogenicity or virulence targets according to claim 4, wherein said nucleic acids areRNAs.

8. Pathogenicity or virulence targets according to claim 5, wherein said nuclesic acids correspond to the nucleic acids of pathogenic organisms comprising Escherichia coli, Salmonellatyphimurium, Klebsiella pneumoniae, Yersiniapestitis, Serratiamarcescens, Haemophilusinfluenzae, Pasteurella multocida, Vibrio cholera, Pseudomonas aeruginosa, Acetinobacter, Moraxellacatarrhalis, Burkholderia pseudomallei, Neisseriameningitidis, Neisseda gonorrhoeae, Campylobacter jejuni, Helicobacter pylori, Bacteroidesfragilis, Clostridium acetobutylicum, Mycobacterium tuberculosis, Streptococcus pyogenes, Streptococcus agalactiae, Staphyloccus aureus and Enterococcus.

9. Pathogenicity or virulence targets according to claim 8 corresponding to nucleic acids of E. coli or Streptococcus agalactiae.

10. Vectors comprising at least one pathogenicity or virulence target according to claim 4.

11. Host cells containing at least one vector according to claim 10.

12. Products of expression of the pathogenicity or virulence targets according to claim 4.

13. Isolated or purified peptides characterized in that they correspond to one of the amino acid sequences SEQ ID Nos. 1 to 15.

14. Isolated or purified peptides according to claim 13 characterized in that they correspond to one of the amino acid sequences SEQ ID Nos 1, 2, 4-15.

15. Antibodies capable of binding specifically to the peptides according to claim 12.

16. Method for inhibiting in vitro the proliferation of a pathogenic microorganism in serum, comprising the use of an effective amount of a compound capable of inhibiting the activity, or of reducing the amount, of pathogenicity or virulence target according to claim 5, or of inhibiting the activity of a peptide selected from SEQ ID Nos: 1, 2 and 4-15.

17. Method for screening compounds capable of inhibiting the expression of the pathogenicity or virulence target according to claim 5, or peptides selected from SEQ ID Nos: 1, 2 and 4-15, comprising bringing into contact with the test compound, demonstrating the possible effect of the compound on their activity, and selecting the active compounds.

18. Method for screening compounds capable of inhibiting the biochemical and/or enzyme activity of the peptides expressed by the athogenicity or virulence target according to claim 5.

19. A method of developing medicinal products for inhibiting a bacterial infection comprising testing the pharmaceutical applicability of compounds screened in the method of claim 18 and found to inhibit.
